(19) United States
(12) Patent Application Publication
(10) Pub. No.: US 2022/0202774 A1
(43) Pub. Date: Jun. 30, 2022

(54) PHARMACEUTICAL COMPOSITION FOR PREVENTION OR TREATMENT OF SPINAL CORD INJURY OR SPINAL STENOSIS

(71) Applicant: NEUROBIT SCIENCE CO., LTD., Gyeonggi-do (KR)

(72) Inventors: Jae Yeol LEE, Seoul (KR); Kyung Tae LEE, Seoul (KR); Tae Young YUNE, Seoul (KR)

(21) Appl. No.: 17/594,889

(22) PCT Filed: Apr. 7, 2020
(86) PCT No.: PCT/KR2020/004696

§ 371 Date: Nov. 2, 2021

(30) Foreign Application Priority Data
Apr. 9, 2019 (KR) 10-2019-0041464
Apr. 6, 2020 (KR) 10-2020-0041361

(51) Int. Cl. A61K 31/4015; A61P 25/00

(52) U.S. Cl. CPC A61K 31/4015; A61P 25/00

(57) ABSTRACT

Proposed is a pharmaceutical composition for prevention or treatment of spinal cord injury or spinal stenosis. The pharmaceutical composition contains 3-(4-chlorophenyl)-4-(4-aminosulfonyl-phenyl)-1-methyl-1H-pyrrole-2,5-dione or a pharmaceutically acceptable salt thereof. It was newly discovered that the compound inhibits activities of pro-inflammatory cytokines and PGE₂, which are inflammatory mediators, thereby suppressing inflammation or pain due to spinal cord injury or spinal stenosis. Since the compound can effectively inhibit inflammation or pain caused by spinal cord injury or spinal stenosis, it is expected that the compound will be useful for the prevention or treatment of spinal cord injury or spinal stenosis.

### TECHNICAL FIELD

The present invention relates to a pharmaceutical composition for prevention or treatment of spinal cord injury or spinal stenosis, the composition containing 3-(4-chlorophenyl)-4-(4-aminosulfonyl-phenyl)-1-methyl-1H-pyrrole-2,5-dione or a pharmaceutically acceptable salt thereof. More particularly, the present invention relates to a pharmaceutical composition which prevents or treats the spinal cord injury or spinal stenosis by inhibiting an inflammatory mediator of the spinal cord injury or spinal stenosis.

### BACKGROUND ART

Spinal stenosis refers to a medical condition in which the spinal canal surrounded by bones and soft tissues which make up the nerve structure narrows. Spinal stenosis causes intermittent claudication, pain in the lower extremities, inability to walk, and the like.

Surgical decompression is a most recommended surgical treatment option as a treatment method for spinal stenosis. However, it is difficult to predict the odds of a successful surgery, and laminectomy may damage anatomical support structures such as muscle fiber ligaments, which may pose a risk of inducing muscular atrophy. Therefore, as a method of treating spinal stenosis, use of a drug which targets a biological material involved in inflammation or pain is preferred.

For spinal stenosis, drugs such as anti-inflammatory drugs, analgesics, and muscle relaxants are commonly used, and steroids may be injected. Among the above, steroids are an adrenocortical hormone with immunosuppressive and strong anti-inflammatory effects and are widely used as a treatment for diseases such as demyelinating diseases of the central nervous system, including multiple sclerosis. However, there are side effects of long-term use of steroids such as weight gain, weakened immunity, increased possibility of infection, peptic ulcer, myopathy, osteonecrosis, cataracts, skin changes, and behavioral disorders.

Spinal cord injury refers to an injury that causes a temporary or permanent change in the function of the spinal cord. The injury causes loss of sensation and loss of muscle function in the parts of the body below the level of injury, pain in the lumbar spine, and the like.

Treatment of spinal cord injury is largely divided into drug treatment and surgical treatment. In the case of drug treatment, administration of a large amount of steroids for acute spinal cord injury has been reported to be effective in recovery. However, since such administration has the same side effects as the side effects of steroid use for spinal stenosis, there are problems with such administration.

Accordingly, coxibs such as celecoxib and rofecoxib may be considered as anti-inflammatory drugs for the treatment of spinal cord injury or spinal stenosis. However, in 2004, coxibs like Merck's rofecoxib were banned due to drastically increased risks of heart attack and stroke with long-term use.

In addition, some studies found that the side effects of rofecoxib on the cardiovascular system may be due to intrinsic chemical properties related to the metabolism of rofecoxib. Accordingly, discovery of a new compound structure is urgently needed, and active research is being carried out to that end (British Journal of Pharmacology (2012) 165, Shin et al.).

### SUMMARY

### Technical Problem

The present inventors conducted research to discover a compound which is effective in prevention or treatment of spinal cord injury or spinal stenosis. As a result, it was found that a composition containing a compound according to the present invention inhibits the expression of pro-inflammatory mediators, which cause inflammation or pain, and thus, the present invention was completed.

Accordingly, the objective of the present invention is to provide a pharmaceutical composition for prevention or treatment of symptoms caused by spinal cord injury or spinal stenosis, the pharmaceutical composition containing a compound of Chemical Formula I or a pharmaceutically acceptable salt thereof.

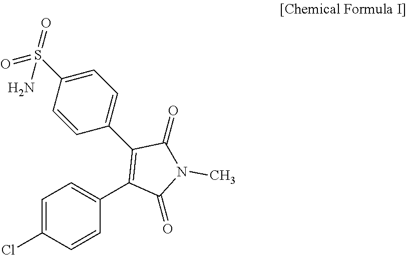 [SMILES: CN1C(=O)C(c2ccc(Cl)cc2)=C(c2ccc(S(N)(=O)=O)cc2)C1=O]

However, objectives of the present invention are not limited to the objective mentioned above. Other unmentioned objectives will be clearly understood by one of ordinary skill in the art on the basis of the description below.

### Technical Solution

In order to accomplish the above objective, the present invention provides

a pharmaceutical composition for prevention or treatment of symptoms caused by spinal cord injury or spinal stenosis, the pharmaceutical composition containing a compound of Chemical Formula I or a pharmaceutically acceptable salt thereof.

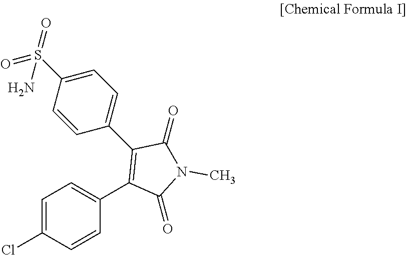 [SMILES: CN1C(=O)C(c2ccc(Cl)cc2)=C(c2ccc(S(N)(=O)=O)cc2)C1=O]

In addition, the present invention provides the pharmaceutical composition of the above, in which the spinal stenosis may be lumbar spinal stenosis.

In addition, the present invention provides the pharmaceutical composition of the above, in which the composition inhibits intermittent claudication, paresis, hypesthesia, paresthesia, sensory disturbance, inflammation, or pain.

In addition, the present invention provides a method of preventing or treating symptoms of spinal cord injury or spinal stenosis, the method including administering to a subject the composition.

In addition, the present invention provides a use of the composition for prevention or treatment of spinal cord injury or spinal stenosis.

### Advantageous Effects

The present inventors newly discovered that a compound containing 3-(4-chlorophenyl)-4-(4-aminosulfonyl-phenyl)-1-methyl-1H-pyrrole-2,5-dione or a pharmaceutically acceptable salt thereof inhibits inflammation or pain due to spinal cord injury and spinal stenosis. Since the compound according to the present invention can effectively inhibit inflammation or pain caused by spinal cord injury or spinal stenosis, it is expected that the compound will be useful for the prevention or treatment of spinal cord injury or spinal stenosis.

### BRIEF DESCRIPTION OF THE DRAWINGS

FIG. 1A shows a target lumbar region. FIG. 1B shows a silicone block used for preparing an animal model. FIG. 1C shows a state in which the silicone block is inserted into the target lumbar region.

FIG. 2 shows the inhibitory effect of the compound according to the present invention on the LPS-induced production of PEG₂.

FIG. 3A shows the results of a rotarod test of a chronic mechanical allodynia-induced animal model and a simulated control group (sham surgery group). FIG. 3B shows the results of measuring the paw withdrawal threshold (PWT) of the chronic mechanical allodynia-induced animal model and the simulated control group (sham surgery group). FIG. 3C shows the results of observing ED-1 positive macrophages in compressed and non-compressed regions after the cauda equina was compressed in the chronic mechanical allodynia-induced animal model.

FIG. 4A shows the PWT measurement results when celecoxib was applied to the chronic mechanical allodynia-induced animal model. FIG. 4B shows the PWT measurement results when the compound according to the present invention was applied to the chronic mechanical allodynia-induced animal model. FIG. 4C shows the expression of TNF-α, interleukin-1β (IL-1β), IL-6, and inducible nitric oxide synthase (iNOS) mRNA 30 minutes after applying celecoxib and the compound according to the present invention to the chronic mechanical allodynia-induced animal model. FIG. 4D shows the results of measuring the relative expression levels of inflammatory mediators when celecoxib and the compound according to the present invention were applied to the chronic mechanical allodynia-induced animal model. FIG. 4E shows the results of measuring the expression level of PEG₂ when celecoxib and the compound according to the present invention were applied to the chronic mechanical allodynia-induced animal model.

### DETAILED DESCRIPTION

Hereinafter, the present invention is described in detail.

The present invention relates to a pharmaceutical composition for treatment or prevention of spinal cord injury or spinal stenosis, the pharmaceutical composition containing a compound of Chemical Formula I or a pharmaceutically acceptable salt thereof.

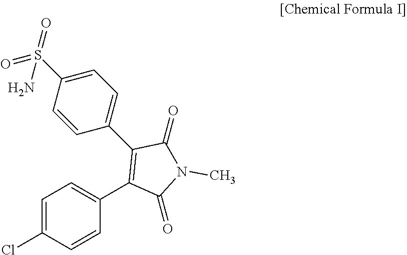 [SMILES: CN1C(=O)C(c2ccc(Cl)cc2)=C(c2ccc(S(N)(=O)=O)cc2)C1=O]

The compound according to the present invention is a derivative of 1H-pyrrole-2,5-dione or 1H-furan-2,5-dione and is named 3-(4-chlorophenyl)-4-(4-aminosulfonyl-phenyl)-1-methyl-1H-pyrrole-2,5-dione.

As used herein, the term “prevention” refers to any action that suppresses or delays the onset of spinal cord injury or spinal stenosis by administration of the pharmaceutical composition according to the present invention.

As used herein, the term “treatment” refers to any action which improves or brings beneficial changes to the symptoms of spinal cord injury or spinal stenosis by administration of the pharmaceutical composition according to the present invention.

As used herein, the term “salt” refers to an acid addition salt formed by a pharmaceutically acceptable free acid. An acid addition salt is obtained from an inorganic acid such as hydrochloric acid, nitric acid, phosphoric acid, sulfuric acid, hydrobromic acid, hydroiodic acid, nitrous acid, or phosphorous acid; or a non-toxic organic acid such as aliphatic monocarboxylate, aliphatic dicarboxylate, phenyl-substituted alkanoate, hydroxyalkanoate, hydroxyalkandioate, aromatic acid, aliphatic sulfonic acid, or aromatic sulfonic acid. Such pharmaceutically non-toxic salts include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, beta-hydroxybutyrate, glycolate, malate, tartrate, methanesulfonate, propanesulfonate, naphthalene-1-sulfonate, naphthalene-2-sulfonate, and mandelate.

The acid addition salt according to the present invention may be prepared by a conventional method, for example, by dissolving the compound in an aqueous solution of excess acid and precipitating the salt in a water-miscible organic solvent such as methanol, ethanol, acetone, or acetonitrile. The acid addition salt may also be prepared by evaporating the solvent or excess acid from the mixture and then drying or by suction filtration of the precipitated salt.

In addition, a pharmaceutically acceptable metal salt may be prepared using a base. For example, an alkali metal salt or alkaline earth metal salt may be obtained by dissolving the compound in a solution containing excess alkali metal hydroxide or alkaline earth metal hydroxide, filtering undissolved compound salt, and evaporating and drying the filtrate. As the metal salt, a sodium, potassium, or calcium salt is pharmaceutically acceptable. A corresponding silver salt may be obtained by reacting an alkali metal salt or alkaline earth metal salt with a suitable silver salt (for example, silver nitrate).

In addition, the compound according to the present invention may include not only pharmaceutically acceptable salts but all salts, isomers, hydrates, and solvates that can be prepared by conventional methods.

The disease targeted in the present invention, “spinal cord injury (SCI)”, refers to a condition in which the spinal cord is also damaged when the spine (central nerve in the spine) becomes damaged due to an accident or disease or a condition in which the spinal cord is damaged due to a disease. Symptoms of spinal cord injury include, but are not limited to, motor and sensory paralysis caused by the failure of proper nerve transmission between the brain and the body.

In addition, the spinal canal is a tube-shaped hollow at the center of the spine. An intervertebral foramen is formed between a lower and upper vertebrae, and the tube-shaped hollow serves as a passageway through which nerves (spinal cord) pass from the brain to the limbs. The shape of the tube is oval or triangular. The tube is the widest in the cervical spine (neck region), narrows in the thoracic spine (chest region), widens again in the lumbar spine (waist region), and then narrows going downward. “Spinal stenosis” is a disease in which narrowing of the spinal canal at the center of the spine, nerve root canal, or intervertebral foramen causes pain in the lower back or multiple neurological symptoms in the legs. Spinal stenosis most commonly occurs in the lumbar region. Therefore, spinal stenosis generally refers to lumbar spinal stenosis. The spinal stenosis of the present invention may be lumbar spinal stenosis but is not limited thereto and includes various types of stenosis related to the spinal canal.

In addition, “lumbar spinal stenosis (LSS)” refers to a disease in which the spinal canal surrounded by bones and soft tissues which make up the nerve structure narrows. Causes of spinal canal stenosis include lumbar spondylolisthesis, slipped disk, ligamentous thickening, and spinal degeneration due to aging. Symptoms of spinal canal stenosis of the present invention include intermittent claudication, pain in the lower extremities, inability to walk, compression of the cauda equina nerve fibers, hypersensitivity, and induction of sensitization of the central nervous system and peripheral nervous system and severe neuropathic pain resulting therefrom, paresis, hypesthesia, paresthesia, sensory impairment, and the like, but are not limited thereto.

Meanwhile, neuroinflammatory responses are known to play an important role in the development and maintenance of spinal neuropathic pain. After tissue damage, immune cells migrate to the site of damage to produce pro-inflammatory cytokines and mediate the inflammatory response. Examples of pro-inflammatory cytokines include TNF-α, IL-1β, IL-6, iNOS, and prostaglandin E2, which sensitizes neuronal pain transmission.

In one example of the present invention, by administering the compound according to the present invention to neuropathic pain-induced rats and observing that the pain threshold is significantly increased in the neuropathic pain-induced rats compared to the vehicle group, it was found that the compound according to the present invention effectively suppressed the pain due to spinal stenosis (refer to Experimental Example 4).

In addition, when the compound according to the present invention was administered at a high dose, it was observed that an analgesic effect lasted until 3 hours after administration. Therefore, it was found that the compound according to the present invention is effective in alleviating chronic mechanical allodynia caused by compression of the cauda equina (refer to Experimental Example 4).

In another example of the present invention, an experiment was performed to observe through RT-PCR whether production of inflammatory mediators was inhibited in rats administered with celecoxib, which is an anti-inflammatory agent, or the compound according to the present invention. As a result of conducting an experiment to observe through ELISA whether production of pro-inflammatory cytokines and PGE₂ was inhibited, expression of TNF-α, IL-1β, IL-6, and iNOS mRNA and production of PGE₂ in the cauda equina were found to be significantly reduced. Therefore, it was found that the compound according to the present invention has an effect of preventing or alleviating inflammation caused by compression of the cauda equina (refer to Experimental Example 5).

The above results demonstrate the utility of the compound according to the present invention in alleviating chronic mechanical allodynia or inflammation, which are symptoms of spinal cord injury or spinal stenosis.

The pharmaceutical composition according to the present invention contains 3-(4-chlorophenyl)-4-(4-aminosulfonyl-phenyl)-1-methyl-1H-pyrrole-2,5-dione or a pharmaceutically acceptable salt thereof as an active ingredient and may also include a pharmaceutically acceptable carrier.

In preparing the pharmaceutical composition, the content of the compound according to the present invention or a pharmaceutically acceptable salt thereof varies depending on the form of the pharmaceutical composition but is preferably in a concentration of 0.01 to 100 wt %. The pharmaceutically acceptable carrier is one that is used commonly during formulation. Examples of the pharmaceutically acceptable carrier include, but are not limited to, saline, sterile water, Ringer's solution, buffered saline, cyclodextrin, dextrose solution, maltodextrin solution, glycerol, ethanol, and liposome. Other conventional additives such as antioxidants or buffers may further be added, if necessary. In addition, diluents, dispersants, surfactants, binders, lubricants, and the like may be additionally added to formulate injectable dosage forms such as an aqueous solution, suspension, or emulsion; pills; capsules; granules; or tablets. Suitable pharmaceutically acceptable carriers and formulation methods may be used depending on the component according to methods disclosed in a paper published by Remington. The pharmaceutical composition according to the present invention is not particularly limited in terms of the dosage form but may be formulated as an injection, an inhalant, an external preparation for skin, or an oral preparation.

The pharmaceutical composition according to the present invention may be administered orally or parenterally (for example, intravenously, subcutaneously, or applied to the skin, nasal passages, or airways) depending on the desired method. Although dosage of the pharmaceutical composition will vary depending on the patient's condition, body weight, severity of disease, dosage form, and route and time of administration, the dose may be appropriately selected by those skilled in the art.

The composition according to the present invention may be administered in a pharmaceutically effective amount. In the present invention, “pharmaceutically effective amount” means an amount sufficient to treat a disease at a reasonable benefit/risk ratio for medical treatment. An effective dose may be determined according to factors including the type and severity of disease of the patient, activity of the drug, sensitivity to the drug, time and route of administration, excretion rate, duration of treatment, and concomitant drugs, and other factors well known in the medical field. The composition according to the present invention may be administered alone or concomitantly with other drugs, may be administered sequentially or simultaneously with conventional drugs, and may be administered in a single dose or multiple doses. In consideration of all of the above factors, it is important to administer an amount that can obtain a maximum effect with a minimum amount without side effects. The amount can be easily determined by those skilled in the art.

Particularly, an effective amount of the composition according to the present invention may vary depending on the age, sex, and weight of the patient. Typically, 0.001 to 150 mg per 1 kg of body weight, preferably 0.01 to 100 mg per 1 kg of body weight, may be administered daily or every other day and administered divided into 1 to 3 administrations a day. However, the dosage may be increased or decreased depending on the route of administration, severity of spinal cord injury or spinal stenosis, sex, weight, age, and the like. Therefore, the dosage does not limit the scope of the present invention in any way.

On the other hand, as another aspect of the present invention, the present invention provides a method of preventing, controlling, or treating spinal cord injury or spinal stenosis, the method including administering to a subject the pharmaceutical composition.

In the present invention, “subject” refers to one in need of a method of preventing, controlling, or treating a disease and more particularly to a human or non-human mammal such as a primate, mouse, rat, dog, cat, horse, or the like.

Hereinafter, preferred examples are provided to aid the understanding of the present invention. However, the examples below are only provided for easier understanding of the present invention, and the contents of the present invention are not limited by the examples.

### EXAMPLES

### Example 1. Preparation for and Method of Experiment

1-1. ¹H NMR Spectra and ¹³C NMR Spectra

In the present experiment, the instrument used for ¹H NMR Spectra and ¹³C NMR Spectra was Bruker Avance DRX 400 (400 MHz) Spectrometer.

In addition, chemical shift (δ) was expressed in ppm using tetramethylsilane (TMS) as an internal standard. Coupling constant (J) was expressed in Hertz (Hz). Signal multiplicity was denoted as singlet (s), doublet (d), triplet (t), quartet (q), broad (br), multiplet (m), or doublet of doublets (dd).

In addition, for low-resolution and high-resolution mass spectra (FABMS, FAB+ energy; 6 kev, emission current: 5 mA, acceleration voltage: 10 kV), JMS-700 Mass Spectrometer (JEOL, JAPAN) was used.

As a TLC plate, a glass plate coated with silica gel (E. Merck Kieselgel 60 F₂₅₄, layer thickness of 0.25 mm) was used. To check for organic compounds on the TLC plate, 254 nm and 365 nm UV light was used, and phosphomolybdic acid (PMA) 5% ethanol solution, p-anisaldehyde 5% ethanol solution, or ninhydrin 5% ethanol solution was used as a color developer.

In addition, Merck Kieselgel 60 Art 9385 (230-400 mesh) was used as silica gel of flash column chromatography, which was performed to separate organic mixtures.

Finally, the reagents required for reaction were mainly purchased from companies such as Sigma-Aldrich, TCI, Acros, and Fluka, and solvents requiring purification were purified by a known method and used.

1-2. Animal Ethics Statement

For experiments related to the present invention, a total of 167 male Sprague-Dawley rats (250 to 270 g, Samtako, Osan, Korea) were used. The rats were kept in an environment of room temperature (23±1° C.) and 60±10% humidity under a 12-hour light/12-hour dark cycle (light on from 07:30 to 19:30) and given free access to water and food. The rats were housed individually in cages (410×282×153 mm, clear polycarbonate) lined with aspen shaving bedding and fed a commercial diet (5L79, PMI Nutrition International, St Louis, Mo.) and a commercial standard feed (Lab Diet 5L791 Purina Mills, Richmond, Ind.). All animal experiments were performed in accordance with the guidelines of the Animal Protection Committee of Kyung Hee University (Permission No. KHUASP(SE)-15-006) and in compliance with the ethical guidelines of the International Association for the Study of Pain.

1-3. Surgery and Compression of Cauda Equina

Compression of the cauda equina was induced on the basis of a previous report [PLoS One (2013) e56580, Ma et al.].

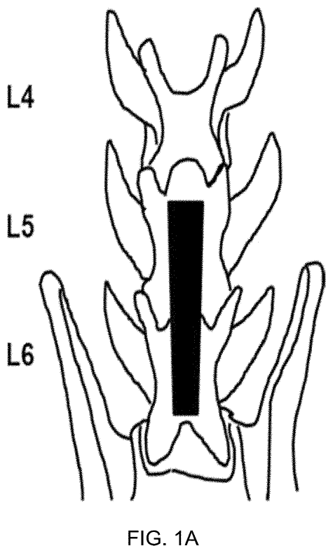

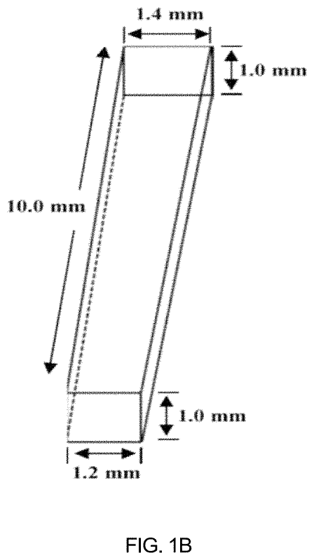

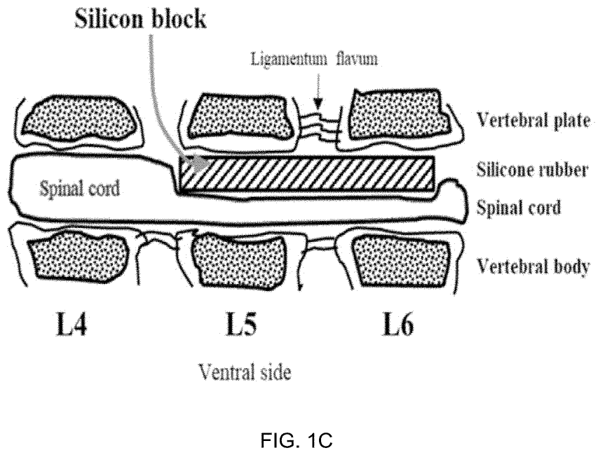

The surgical procedure performed on the rat is as shown in FIG. 1. More specifically, the rats were anesthetized by administering chloral hydrate (500 mg/kg) as an intraperitoneal injection, the backside of each rat was shaved, and the L4 to S2 vertebral plates were exposed.

Next, the ligamenta flava between L4 and L5 were removed. Then, a trapezoidal silicone block (1.00 mm in length×1.2 to 1.3 mm in width×1.00 mm in height) was inserted into the epidural space and placed on the L5 and L6 vertebral plates, and the dural sac was disrupted.

On the other hand, in the simulated control group (sham surgery group), the rats were opened from the posterior and a perforations was made, but no silicone block was inserted.

The body temperature of the rats was maintained at 37±0.5° C. using a heating pad (Biomed S. L., Alicante, Spain) during the surgical procedure. After an injury as described above, the muscles and skin were closed, and the rats were placed in a temperature- and humidity-controlled chamber overnight.

The rats that received surgery were administered subcutaneous supplemental fluids (5 ml, lactated Ringer's solution) and antibiotics (gentamicin, 5 mg/kg, intraperitoneal injection) once a day for 5 days. For all animal models, the body weight and amount of leftover food and water were recorded every morning.

1-4. Behavioral Assessment

Locomotor activity was measured using a rotarod system (Rota ROD-R V2.0, B. S. Technolab Inc.).

More particularly, the rats were placed on a rod with increasing speed from 4 rpm to 40 rpm (accelerated 1 rpm every 5 seconds). Measurement of walking time on the rod until the rats fell off the rod was taken three times for each rat. The rats were acclimated to the rod for 3 minutes at a constant speed of 4 rpm prior to the measurement. The interval between experiments was 20 minutes. For statistical analysis, the average value of three trials was calculated.

Mechanical allodynia was assessed by detecting responses to stimulation with calibrated von Frey filaments and evaluated in terms of paw withdrawal threshold (PWT). The assessment of pain behavior was performed by an experienced researcher who was blind to the experimental conditions.

1-5. Administration of Drug

Only rats with a weight in the range of 350 to 380 g and in which chronic mechanical allodynia (2.5-4.0 g) was induced on the 28th day of compression of the cauda equina were selected as the experimental group. The rats were randomly assigned to three experimental groups treated with a vehicle, celecoxib, or the compound according to the present invention.

Celecoxib (Sigma, St. Louis, Mo.) or the compound according to the present invention was dissolved in methyl pyrrolidone:Tween-80:saline (1:1:8, 100 μl) and injected intraperitoneally at a dose of 2, 5, or 10 mg/kg. The vehicle group was injected with 1-methyl-2-pyrrolidone (1-methyl-2-pyrrolidone:Tween-80:saline (1:1:8)) at an equal dose.

1-6. Preparation of Tissue

At the time of the peak effect (30 minutes after injection of drug), the rats were anesthetized by injecting chloral hydrate (500 mg/kg) and perfused with 0.1 M PBS (pH 7.4), followed by perfusion with a solution containing 4% paraformaldehyde added to PBS.

To make frozen sections, segments of tissue were embedded in OCT and sectioning was performed at 10° C. using a cryostat (CM1850; Leica, Wetzlar, Germany).

For analysis at the molecular level, the rats were perfused with 0.1 M PBS, and a 20 mm-thick section of the cauda equina with the site of injury at the center was isolated and kept frozen at −80° C. until use.

1-7. Immunohistochemistry

The frozen section was immunohistochemically treated with an antibody against ED-1 (CD68, 1:200, Serotec, Raleigh, N.C.) and an antibody against COX-2 (1:100, Abcam, MA). Fluorescence signals were detected by fluorescence microscopy (BX51, Olympus, Japan), and measurement of signal colocalization was performed using MetaMorph software (Molecular Devices, Sunnyvale, Calif.).

1-8. Western Blot

Total protein from the cauda equina segment containing the site of compression was prepared, and western blot analysis was performed. The primary antibodies used for the western blot were as follows: COX-2 (1:1000, Abcam) and β-tublin (1:3000, Sigma).

Quantification of bands was performed using Alphalmager software (Alpha Innotech Corporation, San Leandro, Calif.).

1-9. RNA Isolation and RT-PCR

RT-PCR of TNF-α, IL-1β, IL-6, iNOS, and GAPDH was performed. Primers for each sequence are shown in Table 1 below (5′->3′).

TABLE 1

| No. | Name | forward | reverse |
|---|---|---|---|
| 1 | TNF-α | 5′-CCC AGA CCC TCA CAC TCA GAT-3′ | 5′-TTG TCC CTT GAA GAG AAC |
| | | | CTG-3′ |
| 2 | IL-1β | 5′-GCA GCT ACC TAT GTC TTG CCC | 5′-GTC GTT GCT TGT CTC TCC TTG |
| | | GTG-3′ | TA-3′ |
| 3 | IL-6 | 5′-AAG TTT CTC TCC GCA AGA TAC | 5′-AGG CAA ATT TCC TGG TTA TAT |
| | | TTC CAG CCA-3′ | CCA GTT-3′ |
| 4 | COX-2 | 5′-CCA TGT CAA AAC CGT GGT GAA | 5′-ATG GGA GTT GGG CAG TCA |
| | | TG-3′ | TCA G-3′ |
| 5 | iNOS | 5′-CTC CAT GAC TCT CAG CAC AGA G- | 5′-GCA CCG AAG ATA TCC TCA |
| | | 3′ | TGA T-3′ |
| 6 | GAPDH | 5′-AAC TTT GGC ATT GTG GAA GG-3′ | 5′-GGA GAC AC CTG GTC CTC AG- |
| | | | 3′ |

1-10. Measurement of PGE₂ Level

Levels of PEG₂ in the cauda equina fibers were analyzed using a PEG₂ ELISA kit (Monoclonal, Cayman Chemical Ann Arbor, Mich.) according to the manufacturer's instructions.

1-11. Statistical Analysis

The data are expressed as Mean±SD or SEM.

Comparison between experimental groups was evaluated for statistical significance using an unpaired student t test. Multiple comparisons between groups were performed using a one-way ANOVA.

Some behavioral assessment scores were analyzed using repeated measures ANOVA. Dunnett's multiple comparison was used for post hoc analysis.

A group size was expressed as the number of animals in each group. Statistical significance was accepted when p was less than 0.05 (p<0.05). All statistical analyses were performed using SPSS 15.0 (SPSS Science, Chicago, Ill.).

### Example 2. Preparation of 3-(4-Chlorophenyl)-4-(4-Aminosulfonyl-Phenyl)-1-Methyl-1H-Pyrrole-2,5-Dione, Compound According to Present Invention

2-1. Preparation of 2-(4-(Chlorosulfonyl)Phenyl)Acetic Acid

20 ml of CISO₃ was cooled in an ice bath. After slowly adding phenylacetic acid (5.00 g, 36.72 mmol) to the CISO₃, the ice bath was removed, and the temperature of the solution was raised to room temperature. Then, the solution was stirred for 12 hours. Upon completion of the reaction, the solution was slowly dropped into ice water to remove any remaining CISO₃H. The result from the above was filtered to obtain 2-(4-(chlorosulfonyl)phenyl)acetic acid, which is a white solid product (7.84 g, 91%).

¹H-NMR (400 MHz, DMSO-d₆) δ: 11.6 (1H, s), 7.56 (2H, d, J=8.4 Hz), 7.23 (2H, d, J=8.4 Hz), 3.58 (2H, s).

2-2. Preparation of 2-(4-Sulfamoylphenyl)Acetic Acid

In an ice bath, the 2-(4-(chlorosulfonyl)phenyl)acetic acid (2.00 g, 8.55 mmol) obtained in Example 2-1 was dissolved in anhydrous MeOH and cooled. An excess amount of NH₄OH (25%) was added dropwise to the mixture, and the ice bath was removed. Then, the temperature of the solution was raised to room temperature, and the solution was stirred for 12 hours. Upon completion of the reaction, HCl was added for acidification, and the solution was stirred under reduced pressure for 12 hours. After the completion of the reaction, HCl was added for acidification, and the solvent was removed under reduced pressure. Then, extraction was performed using EtOAc, an organic layer was dried over anhydrous MgSO₄, and the solvent was removed under reduced pressure. A reaction mixture obtained from the organic layer was crystallized using ACN and DCN to obtain 2-(4-sulfamoylphenyl)acetic acid, which is a white solid product (1.03 g, 56%).

¹H-NMR (400 MHz, DMSO-d₆) δ: 7.75 (2H, d, J=8.4 Hz), 7.44 (2H, d, J=8.4 Hz), 7.34 (2H, s), 3.68 (2H, s).

2-3. Preparation of Ethyl 2-(4-Methylphenyl)-2-Oxoacetate

Chlorobenzene (2.00 g, 17.77 mmol) was dissolved in anhydrous DCM under anhydrous conditions and cooled to −5° C. using a low temperature reactor. Anhydrous AlCl₃ (2.1 g, 16.28 mmol) and ethyl chlorooxoacetate (0.6 ml, 5.43 mmol) were slowly added dropwise to the mixture. After stirring for 4 hours at 0° C., the reaction was terminated by placing the resulting product on ice. After extraction with DCM, an organic layer was washed with distilled water. Then, the organic layer was dried over anhydrous MgSO₄ and filtered under reduced pressure. Once again under reduced pressure, the solvent was removed to obtain ethyl 2-(4-methylphenyl)-2-oxoacetate, which is a pale yellow liquid product (3.01 g, 80%).

¹H-NMR (400 MHz, CDCL₃-d) δ: 7.99 (2H, d, J=8.4 Hz), 7.49 (2H, d, J=8.4 Hz), 4.45 (2H, q, J=7.2 Hz), 1.43 (3H, t, J=7.2 Hz).

2-4. Preparation of 2-(4-Chlorophenyl)-2-Oxoacetic Acid

The ethyl 2-(4-methylphenyl)-2-oxoacetate (0.26 g, 1.23 mmol) obtained in Example 2-3 was dissolved in DCM and 2N NaOH was added in excess, followed by stirring at room temperature for 3 hours. Upon completion of the reaction, HCl was added for acidification, and extraction was performed with DCM. An organic layer was dried over anhydrous MgSO₄, and the solvent was removed under reduced pressure. A reaction mixture obtained from the organic layer was crystallized using DCM and hexane to obtain 2-(4-chlorophenyl)-2-oxoacetic acid, which is a white solid product (0.14 g, 60%).

¹H-NMR (400 MHz, CDCL₃-d) δ: 8.31 (2H, d, J=8.8 Hz), 7.53 (2H, d, J=8.8 Hz)

2-5. Preparation of 3-(4-Chlorophenyl)-4-(4-Aminosulfonyl-Phenyl)-1-Methyl-1H-Pyrrole-2,5-Dione

The 2-(4-sulfamoylphenyl)acetic acid (0.10 g, 0.46 mmol) obtained in Example 2-2 and 2-(4-chlorophenyl)-2-oxoacetic acid (0.09 g, 0.46 mmol) obtained in Example 2-4 were dissolved in Ac₂O and stirred for 8 hours while refluxing at 85° C. When the reaction was completed, the solvent was removed at a high temperature under reduced pressure. After the reaction mixture was dissolved in EtOH, CH₃NH₂(33%) (0.38 ml, 3.74 mmol) was added in excess and stirred for 18 hours. After the reaction was completed, the solvent was removed under reduced pressure, and extraction was performed with EtOAc. An organic layer was dried over anhydrous MgSO₄, and the solvent was removed under reduced pressure. A product obtained by column chromatography was crystallized using IPE, and from this, 3-(4-chlorophenyl)-4-(4-aminosulfonyl-phenyl)-1-methyl-1H-pyrrole (0.05 g, 28%), which is a pale yellow solid product, was obtained.

¹H-NMR (400 MHz, DMSO-d₆) δ: 7.86 (2H, d, J=8.4 Hz), 7.56 (2H, d, J=8.4 Hz), 7.53 (2H, d, J=8.4 Hz), 7.45 (2H, br), 7.41 (2H, d, J=8.4 Hz), 3.04 (3H, s).

¹³C-NMR (100 MHz, Acetone-d₆) δ: 171.35, 171.29, 145.82, 138.13, 137.06, 136.39, 133.39, 132.58, 131.36, 129.64, 128.37, 127.05, 23.35.

Table 2 below shows the structure and ¹H-NMR results of the final product 3-(4-chlorophenyl)-4-(4-aminosulfonyl-phenyl)-1-methyl-1H-pyrrole-2,5-dione and the product of each step of Example 2 performed to prepare the final product.

TABLE 2

| No. | Name | Structure | Spectroscopic data |
|---|---|---|---|
| Example 2-1. | 2-(4(chlorosulfonyl)phenyl)acetic acid | 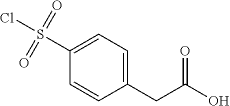 [SMILES: O=C(O)Cc1ccc(S(=O)(=O)Cl)cc1] | ¹H-NMR (400 MHz, DMSO-d₆) δ: 11.6 (1H, s), 7.56 (2H, d, J = 8.4 Hz), 7.23 (2H, d, J = 8.4 Hz), 3.58 (2H, s). |
| Example 2-2. | 2-(4-sulfamoylphenyl)acetic acid | 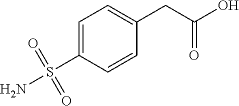 [SMILES: NS(=O)(=O)c1ccc(CC(=O)O)cc1] | ¹H-NMR (400 MHz, DMSO-d₆) δ: 7.75 (2H, d, J = 8.4 Hz), 7.44 (2H, d, J = 8.4 Hz), 7.34 (2H, s), 3.04 (2H, s). |
| Example 2-3. | ethyl 2-(4-chlorophenyl)-2- oxoacetate | 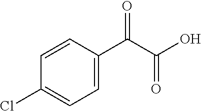 [SMILES: O=C(O)C(=O)c1ccc(Cl)cc1] | ¹H-NMR (400 MHz, DMSO-d₆) δ: 7.99 (2H, d, J = 8.4 Hz), 7.49 (2H, d, J = 8.4 Hz), 4.45 (2H, q, J = 7.2 Hz), 1.43 (3H, t, J = 7.2 Hz). |
| Example 2-4. | 2-(4-chlorophenyl)-2-oxoacetic acid | 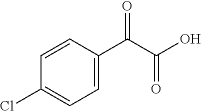 [SMILES: O=C(O)C(=O)c1ccc(Cl)cc1] | ¹H-NMR (400 MHz, DMSO-d₆) δ: 8.31 (2H, d, J = 8.8 Hz), 7.53 (2H, d, J = 8.8 Hz) |
| Example 2-5. | 3-(4-chlorophenyl)-4-(4- aminosulfonyl-phenyl)-1- methyl-1H-pyrrol-2,5-dione | 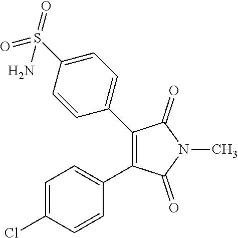 [SMILES: CN1C(=O)C(c2ccc(Cl)cc2)=C(c2ccc(S(N)(=O)=O)cc2)C1=O] | ¹H-NMR (400 MHz, DMSO-d₆) δ: 7.86 (2H, d, J = 8.4 Hz), 7.56 (2H, d, J = 8.4 Hz), 7.53 (2H, d, J = 8.4 Hz), 7.45 (2H, br), 7.41 (2H, d, J = 8.4 Hz), 3.04 (3H, s). ¹³C-NMR (100 MHz, Acetone-d₆) δ: 171.35, 171.29, 145.82, 138.13, 137.06, 136.39, 133.39, 132.58, 131.36, 129.64, 128.37, 127.05, 23.35. |

### Experimental Example 1: Biological Evaluation of 3-(4-Chlorophenyl)-4-(4-Aminosulfonyl-Phenyl)-1-Methyl-1H-Pyrrole-2,5-Dione

Screening for Physiological Activity

The physiological activity of 3-(4-chlorophenyl)-4-(4-aminosulfonyl-phenyl)-1-methyl-1H-pyrrole-2,5-dione prepared according to Example 2 has been expressed in terms of IC₅₀, which is the concentration at which there is 50% inhibition of the activity of an enzyme. On the other hand, for the cytotoxicity of the compounds according to the present invention, cell viability values (IC₅₀) were measured using the MTT assay.

1-1. Screening for Inhibition of Production of LPS-induced PGE₂ by Macrophage Cell Line

RAW 264.7 (a murine macrophage cell line) was obtained from Korea Cell Line Bank (KCLB). RAW 264.7 was cultured in Dulbecco's Modified Eagle Medium (DMEM) containing 10% fetal bovine serum (FBS), penicillin (100 units/mL), and streptomycin sulfate (100 μg/mL) at 37° C. and in a humidified atmosphere of 5% carbon dioxide in air.

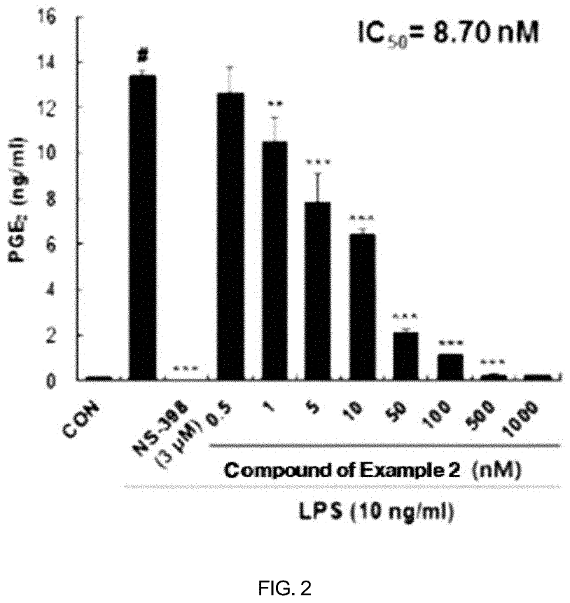

Using DMEM, 1 mL each of RAW 264.7 with a concentration of 5×10⁵ cells/mL was seeded in 24 wells. The seeded cells were left overnight, the medium was changed, and then the drug was treated at an appropriate concentration. After incubating for 1 hour, LPS was treated at 1 μg/ml, followed by incubation for 24 hours (or an appropriate length of time). The supernatant was taken and diluted 5-fold. 150 μl of an assay buffer was added to Non-Specific Binding (NSB) wells, and 100 μl of the assay buffer was added to zero standard (Bo) wells. 100 μl of standard sample and 50 μl of PGE₂ conjugates were added to the remaining wells (excluding the NSB wells). 50 μl of PGE₂ antibody solution was added to the wells and shaken for 2 hours. The contents of each well were suctioned off, and the wells were washed 5 times with wash buffer. 200 μl of para-nitrophenyl phosphate (pNPP) substrate was added to all wells. After storing the wells at room temperature (in a clean bench) for 1 hour, 50 μl of a stop solution was added, and absorbance was measured at 405 nm. The amount of PGE₂ production was quantified using the measured absorbance value and a standard curve, and the 50% inhibitory concentration (IC₅₀) was obtained by comparing with the group treated with LPS alone. The results are shown in Table 3 below. In addition, as shown in FIG. 2, it was confirmed that the IC₅₀ value for inhibition of PGE₂ production was 5.95 nM (positive control: NS-398, 3 μM). This means that the inhibitory effect of 3-(4-chlorophenyl)-4-(4-aminosulfonyl-phenyl)-1-methyl-1H-pyrrole-2,5-dione on LPS-induced biosynthesis of PEG₂ stands at 8.70 nM (IC₅₀).

1-2. Measurement of Cytotoxicity of Macrophages

RAW 264.7 (the murine macrophage cell line) was cultured in DMEM containing 10% FBS, penicillin (100 units/ml), and streptomycin sulfate (100 μg/ml) at 37° C. and in a humidified atmosphere of 5% carbon dioxide in air. Cells were collected using centrifugation and a scraper. The cells were added at a concentration of 1×10⁵ cells/well to wells of a 96-well plate containing Roswell Park Memorial Institute (RPMI) 1640 medium, which includes 10% FBS. 3 beta, 4 beta-epoxy-8a-isobutyryloxyguaia-1(10),11, (13)-diene-12.6a-olide was dissolved in methylsulfoxide (DMSO) as a solvent. In all experiments, the concentration of DMSO did not exceed 0.1%. After one night, samples and LPS (1 μg/ml) were added to the wells, and the plate was incubated for 24 hours. After washing the cells once, 50 μl of a medium free of FBS and containing MTT [(3-(4,5-dimethylthiazol-2-yl)-2,5-diphenyltetrazolium bromide] at a concentration of 5 mg/ml was added. After incubation at 37° C. for 4 hours, the medium was removed. Then, formazan blue formed in the cells was dissolved in 100 μl of DMSO, and absorbance of the dissolution was measured at 540 nm to determine the cytotoxic effect with an IC₅₀ value. IC₅₀ refers to the concentration at which the number of cells is reduced by 50% compared to when no compound is treated.

The cytotoxicity of 3-(4-chlorophenyl)-4-(4-aminosulfonyl-phenyl)-1-methyl-1H-pyrrole-2,5-dione is shown in Table 3 below.

TABLE 3

| | | IC₅₀(μM) |
|---|---|---|
| Compound | Cell viability(μM) | PGE₂ |
| 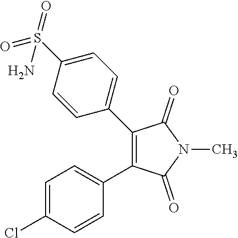 [SMILES: CN1C(=O)C(c2ccc(Cl)cc2)=C(c2ccc(S(N)(=O)=O)cc2)C1=O] | >10 | 0.0087 |
| Celecoxib | | 0.008 |

*Value from reference literature: Bioorg. Med. Chem. Lett. (2012), Kaur, J. et al.).

### Experimental Example 2: Confirmation of Induction of Mechanical Allodynia Using Silicone Block

First, using the rats that were operated on in Example 1-3, it was examined whether chronic neuropathic pain occurred in the mice after the compression of the cauda equina. Motor and sensory tests were performed pre-operation and at predetermined time points between 1 and 28 days after surgery.

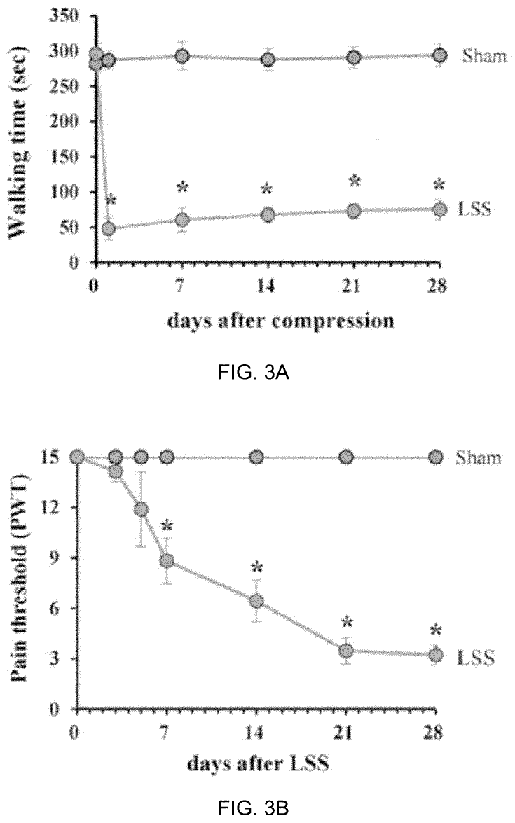

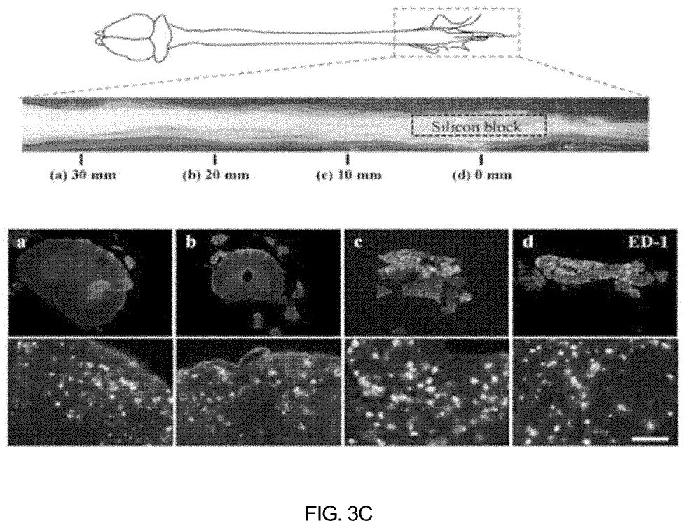

As a result, as shown in FIG. 3A, all animal models were able to walk on the rotarod for 289±15 seconds before surgery. However, on the first day following the compression of the cauda equina, latency for a fall off the rotarod decreased significantly to 48.2±16 seconds. In addition, due to progress of natural recovery, walking efficiency on the 28th day after the compression of the cauda equina increased slightly to 75.2±14 seconds.

On the other hand, as shown in FIG. 3B, the PWT in response to innocuous mechanical stimuli gradually decreased starting on the third day after the compression of the cauda equina, while significant tactile allodynia was observed until the 28th day (PWT 3.22±0.6).

In addition, in the stimulated control group (sham surgery group), there was no change in the walking time on the rotarod or the tactile withdrawal threshold.

### Experimental Example 3: Confirmation of Macrophage Infiltration Due to Compression of Cauda Equina

Infiltration of inflammatory cells is a response to a damage to the nervous system. The infiltration induces not only activation of resident immune cells but also production and secretion of various inflammatory mediators such as pro-inflammatory cytokines and PGE₂. The inflammatory mediators can promote neuroimmune activation and sensitize primary afferent nerve cells, thereby causing hypersensitivity to pain.

Accordingly, in order to see whether an inflammatory mediator is expressed at a site of macrophage infiltration, an experiment in which the site of macrophage infiltration was observed was performed.

In addition, an experiment was performed to observe spatial patterns of infiltrating macrophages in areas other than the spinal cord and cauda equina by immunostaining the ED-1 antibody on the 28th day after the compression of the cauda equina.

As a result, as shown in FIG. 3C, macrophage infiltration was confirmed in the spinal cord tissue and cauda equina nerve fibers on the 14th day after the compression of the cauda equina.

More particularly, ED-1 positive macrophages could be identified at the compression site of the cauda equina. Moreover, the infiltrating macrophages could be observed in uncompressed sites and a bundle of nerve fibers on the dorsal side of the spinal cord (the dorsal funiculus), which is 30 mm away from the lesion epicenter.

### Experimental Example 4: Confirmation of Compound According to Present Invention Alleviating Chronic Mechanical Allodynia Induced by Lumbar Spinal Stenosis

To investigate the effect of the compound according to the present invention in chronic mechanical allodynia, an anti-inflammatory drug celecoxib (2, 5, 10 mg/kg, intraperitoneal injection) was administered to chronic mechanical allodynia-induced rats on the 28th day after injury.

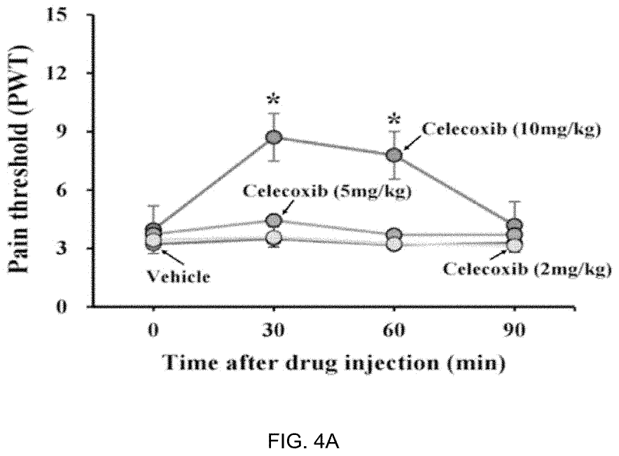

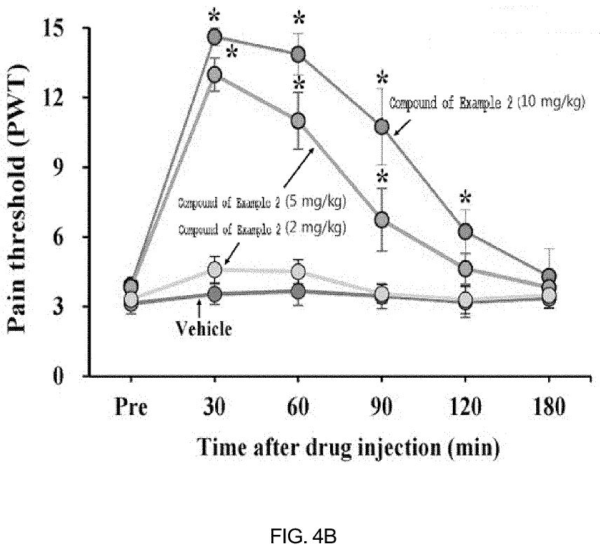

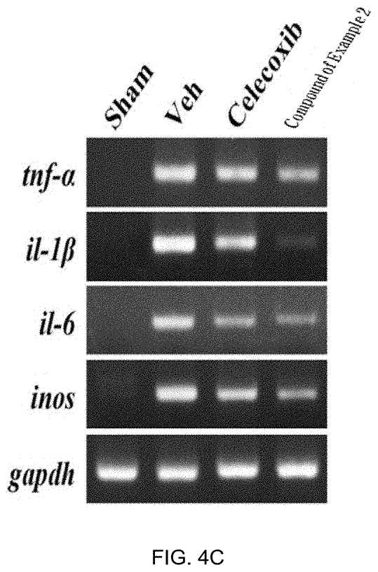

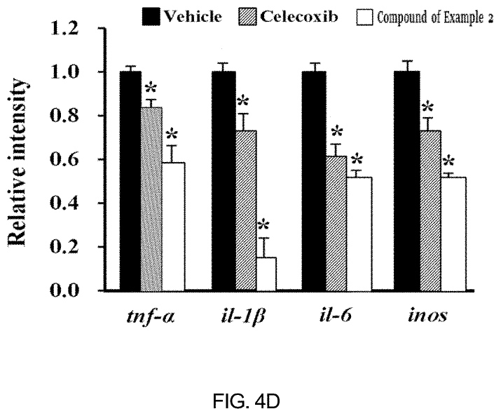

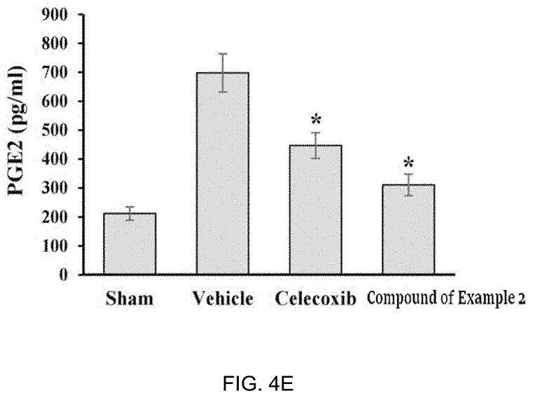

As a result, as shown in FIG. 4A, it was confirmed that the PWT 30 and 60 minutes after injecting celecoxib (10 mg/kg) increased significantly compared to the PWT of the vehicle group.

In addition, the compound according to the present invention (2, 5, 10 mg/kg, intraperitoneal injection) was administered to chronic mechanical allodynia-induced rats.

As a result, as shown in FIG. 4B, it was confirmed that the PWT 30 minutes after injecting the compound according to the present invention increased significantly in a dose-dependent manner compared to the PWT of the vehicle group.

In addition, when the compound according to the present invention was administered in a high dose, the analgesic effect was maintained for 3 hours. From these results, it was confirmed that the compound according to the present invention can alleviate chronic mechanical allodynia that occurs after the compression of the cauda equina.

### Experimental Example 5: Confirmation of Compound According to Present Invention Inhibiting Expression of Inflammatory Mediators

An experiment was performed using rats administered with celecoxib and the compound according to the present invention to confirm through RT-PCR whether production of inflammatory mediators is inhibited. Another experiment was performed to confirm through ELISA whether production of PGE₂ is inhibited.

As a result, as shown in FIGS. 4C and 4D, it was confirmed that 30 minutes after administering celecoxib or the compound according to the present invention to chronic mechanical allodynia-induced rats, expression of TNF-α, IL-6, and iNOS mRNA was significantly reduced.

In addition, as shown in FIG. 4E, when compared with the stimulated control group (sham surgery group), the level of PEG₂ production in the cauda equina of the chronic mechanical allodynia-induced rats was significantly increased by the compression of the cauda equina.

On the other hand, it was confirmed that administering celecoxib or the compound according to the present invention to chronic mechanical allodynia-induced rats significantly reduced PGE₂ production in those rats compared to the vehicle group.

The description of the present invention stated above is only for illustrative purposes. Those skilled in the art will appreciate that various alternatives, modifications, and equivalents are possible without changing the spirit or essential features of the present invention. Therefore, the examples described above are intended to be illustrative in all aspects and should not be construed as being restrictive.

### INDUSTRIAL APPLICABILITY

The present invention provides a technology for developing a composition for the prevention or treatment of spinal cord injury or spinal stenosis through inhibition of pro-inflammatory cytokines and PGE₂. Accordingly, the present invention can provide a composition for alleviation of inflammation and pain, which does not have the same problems as conventional surgeries and steroid use. The technology according to the present invention may be used widely in the field of preventing or developing a drug for spinal cord injury and spinal stenosis.

1. A method of preventing or treating spinal cord injury or spinal stenosis, the method comprising:
administering to a subject a composition comprising a compound of Chemical Formula I or a pharmaceutically acceptable salt thereof:

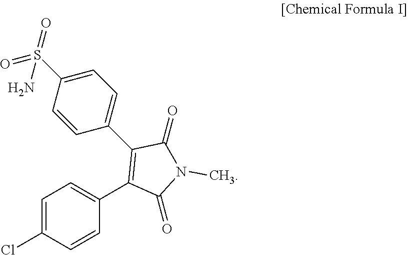 [SMILES: CN1C(=O)C(c2ccc(Cl)cc2)=C(c2ccc(S(N)(=O)=O)cc2)C1=O]

2. The method of claim 1, wherein the spinal stenosis is lumbar spinal stenosis.

3. The method of claim 1, wherein the composition inhibits intermittent claudication, paresis, hypesthesia, paresthesia, sensory disturbance, inflammation, or pain.

4.-5. (canceled)
